(19) United States
(12) Patent Application Publication
(10) Pub. No.: US 2014/0276019 A1
(43) Pub. Date: Sep. 18, 2014

(54) PET Imaging With Partially Radiation-Transparent Probes-Inserts

(71) Applicants: Stanislaw Majewski, Morgantown, WV (US); Alexander Stolin, Morgantown, WV (US)

(72) Inventors: Stanislaw Majewski, Morgantown, WV (US); Alexander Stolin, Morgantown, WV (US)

(21) Appl. No.: 13/827,084

(22) Filed: Mar. 14, 2013

(51) Int. Cl. A61B 6/03

(52) U.S. Cl. CPC A61B 6/037
USPC 600/426; 600/425

(57) ABSTRACT

A probe is disclosed having a positron emission tomography sensor. An imaging system is provided having the probe and at least one external positron emission tomography detector and a data acquisition computer system for collecting data simultaneously from said positron emission sensor of said probe and said positron emission tomography detector. A method for evaluating a target organ of a patient utilizing the probe and imaging system, and performing a biopsy of the organ is disclosed.

### GOVERNMENT INTEREST

This invention was supported in part by the Department of Defense, Army under Federal Grant No. W81XWH-09-1-0420. The government may have certain rights in this invention.

### CROSS-REFERENCE TO RELATED APPLICATIONS

Not applicable.

### BACKGROUND OF THE INVENTION

1. Field of the Invention

The present invention relates to a device and method for performing imaging of organs and tumors. More particularly, an endorectal positron emission tomography (PET) probe is provided for imaging of a prostate gland wherein the PET probe (device) is combined with PET imaging detectors.

2. Description of the Background Art

There are >200,000 new cases and nearly 30,000 deaths each year from prostate cancer (PCa) Prostate-specific antigen (PSA) testing has allowed early detection of impalpable PCa. Early detection has lowered the incidence of advanced disease with extracapsular extension and the subsequent early treatment appears to improve survival rate. After anomalous PSA results, the patient undergoes biopsy, and if the biopsy is positive, the patient undergoes surgery. The main objective of surgery is to remove the cancer at lowest functional cost, i.e. preserving continence and sexual function. The stage of the cancer decides the limits of the resection, and the larger the tumor the wider the excision needed, with radical prostatectomy as the limit of standard treatment. An accurate localization of the tumor and assessment of its size has two important advantages: it can direct the biopsy and can assist with the surgery. Biopsy results may be negative despite the presence of cancer due to sampling error. Prostate cancer is the only human cancer that does not have a standard method to image the primary tumor. The “blind” biopsy typically performed today under ultrasound (US) guidance results in high false negative diagnosis with many missed cancers. Accurate localization of the tumor, within the prostate and pelvic region, will better enable a tumor-free margin. Such accurate assessment today is not available with conventional imaging techniques [ultrasound (US), computed tomography (CT), magnetic resonance imaging and PET]. Standard PET scanners have spatial resolution inadequate to meet the clinical needs of prostate imaging—particularly when using specific, targeted imaging agents.

The diagnosis of prostate cancer is commonly based on a combination of digital rectal examination (DRE), serum prostate specific antigen (PSA) value, and transrectal ultrasound (TRUS) guided prostate biopsy findings. Conventional blind “biopsy” procedures under Tissue Differentiating Ultrasound are able to visualize only the structure and the margins of an organ, and thus do not provide differentiation between a cancerous tissue and healthy tissue.

Prostate cancer is the only human cancer that does not have a standard reliable method of imaging of the primary tumor. Functionally blind biopsy typically performed today under transrectal ultrasound guidance results in high false negative diagnoses with many missed cancers. Accurate localization of the tumor, within the prostate and pelvic region, will allow definition of a tumor-free margin. Such accurate assessment is generally not available in the present state of the art, with the conventional imaging techniques available to urologists.

The main problem is that prostate cancer is difficult to visualize in its early stage using current imaging technology. Conventional imaging modalities, such as ultrasound, CT (computed tomography) scan, and MRI (magnetic resonance imaging), can be used for the anatomic evaluation of prostate cancer. However, visible anatomic changes are not always present in early stages of the disease, making the use of current imaging modalities difficult in early detection of prostate cancer sites. The key problem with conventional guiding systems during prostate biopsy is that they are based on symmetrical anatomical sampling of the prostate, and not on the location of the cancer. The main challenge continues to be the inability to visualize the cancer in its early stages using current imaging technology.

U.S. Pat. No. 7,894,876 “Combined MR-optical coil for prostate, cervix, and rectum cancer imaging diagnostics” discloses a combined MR and optical system that may be used to guide a biopsy.

U.S. Pat. No. 7,711,409 “Opposed view and dual head detector apparatus for diagnosis and biopsy with image processing methods” discloses opposed gamma cameras for guiding a biopsy needle, but discloses no ultrasound imaging components.

U.S. Pat. No. 7,653,427 “Method and instrument for minimally invasive sentinel lymph node location and biopsy” discloses a radiation detector coupled with an ultrasound probe, for locating the position of a tagged tissue, and placement of a biopsy device.

U.S. Pat. No. 6,951,542 “Method and apparatus for ultrasound imaging of a biopsy needle or the like during an ultrasound imaging examination” discloses method including imaging and injection of contrast agents for placement of a biopsy device.

U.S. Pat. No. 6,546,279 “Computer controlled guidance of a biopsy needle” discloses a system for guiding a biopsy needle using one or more of computed tomography imaging, magnetic resonance, fluoroscopic imaging, or 3-D ultrasound imaging.

U.S. Pat. No. 6,512,943 “Combined ultrasound-radionuclide device for percutaneous ultrasound-guided biopsy and method of use” discloses a system and apparatus for performing tissue biopsy. An ultrasound imager and a “radionuclide detectors” are used, external to a patient, to locate “nuclear medicine tracer uptake” in the patient and generate superimposed images of an area of interest.

U.S. Pat. No. 5,776,062 “Enhanced breast imaging/biopsy system employing targeted ultrasound” discloses a system using X-ray imaging and ultrasound, external to a patient, to provide 3-D imaging of an area of interest for use with a biopsy procedure.

U.S. Pat. No. 5,170,055 “Radiation detecting biopsy probe” discloses a handheld biopsy probe that is guided by means of a scintillation crystal, but uses no ultrasound imaging. The device is used externally on a patient, as the primary application is for the detection of tumors in lymph nodes.

U.S. Pat. No. 5,014,708 “Radioactive ray detecting therapeutic apparatus” discloses a “radioactive ray guided” therapeutic device, where in one embodiment, the delivered therapy comprises destroying target cells by ultrasound, and removal of the cells by aspiration.

U.S. Pat. No. 4,995,396 “Radioactive ray detecting endoscope” discloses an endoscope having both an ultrasonic imaging device and a radioactive ray (e.g., beta radiation) detecting device in the tip of the endoscope, but does not disclose use of a biopsy device.

U.S. Pat. No. 4,781,198 “Biopsy tracer needle” discloses a method and device for obtaining a tissue sample, comprising a biopsy tracer needle (i.e., containing a radiation source) guided to a target tissue by means of an external scintillation device. No use of ultrasound is disclosed.

U.S. Published Application No. 2009/0270760 “Biopsy devices” discloses a biopsy device utilizing an isotope-tagged needle mounted to a cradle support mechanism, where PET scanning is used to position the needle in a target tissue by manipulation of the cradle. No use of ultrasound is disclosed.

U.S. Published Application Serial No. 2007/0282221 “Ultrasound assist and X-ray assist biopsy devices” discloses a biopsy table, where a biopsy needle may be directed to a targeted tissue area by using an X-ray guided procedure for locating micro-calcifications, and using an ultrasound guided procedure for locating lesion masses.

U.S. Published Application Serial No. US 2010/0198063 A1 “Multi-Modality Of Phantoms And Methods For Co-Registration Of Dual PET-Transrectal Ultrasound Prostate Imaging” discloses use of a PET scanner and a transrectal ultrasound (TRUS) probe. The TRUS probe is inserted into the rectum of a patient for acquiring a TRUS image data of the prostate stepwise and then moving the patient bed to position the point sources near the external PET-center and acquiring the image, and then superimposing the PET image with the TRUS image for gaining a resulting image showing an anatomical and functional detail.

What is needed is a probe, and more specifically a prostate PET endorectal probe, and an imaging system, and method of evaluating a target organ of a patient, which overcomes the shortcomings of the present state of the art. Currently, imaging is performed separately with and without the PET probes, and the imaging results are combined. There is currently no quick method for combining images from the PET scanner and PET probes. Additionally, the presence or removal of and endorectal probe changes the position of the prostate, which can make difficult of invalidate the co-registration of the two imaging sessions. There is a need for simultaneous imaging to overcome this problem.

Typical PET detector modules, inserts, or probes are currently designed to absorb maximum fraction of annihilation 511 keV gamma rays impinging on them. Thus, in current images acquired using PET probes, the PET probe is seen as an opaque object hiding the PET image details of the tissue sections in front and behind the probe in the line of view. There is a need for a PET scanning system that includes a PET probe that is sufficiently transparent to allow visibility of the tissue in front or behind the probe.

### BRIEF SUMMARY OF THE INVENTION

The present invention fulfills the long an unmet needs of the health care clinician in evaluating a target organ of a patient. The present invention is an improvement over current PET imaging because the present invention allows for simultaneous imaging using the PET imager and the high-resolution small compact probe(s) placed strategically inside the imaging volume of the PET scanner. The probe is partially transmissive to a substantial fraction of the 511 keV annihilation gamma rays, which allows the rays to be detected by the PET scanner with the probe effectively “ignored.” The combination of the PET imager with the PET probe(s) provides higher resolution imaging of the limited region of interest in the broader imaging scene provided by the PET scanner alone. Only the stopped fraction of the annihilation gammas in the probe contributes to the imaging using the probe. As both imaging modes are performed simultaneously, there is the ability to make almost automatic co-registration of the two images. Co-registration or fusion of the two imaging sets of data is crucial in providing useful information and guidance to the physician.

This invention provides a probe comprising a housing having an external shell and an interior space and a positron emission tomography sensor located within the interior space of the housing. This probe is partially transmissive to a substantial fraction of 511 keV annihilation gamma rays.

In a preferred embodiment, the positron emission tomography sensor of the probe is disposed within said interior of said housing such that it is rotatable about an axis of rotation.

In another embodiment, the probe includes a positron emission photoarray and a positron emission detection electronics each in juxtaposition to and in communication with the positron emission sensor.

In yet another embodiment, the probe includes an electronic sensor positioning system located either on the exterior of the housing of the probe or within the interior of the housing, and the electronic sensor positioning system is in communication with an outside positron emission tomography imager.

In another embodiment, the probe includes an external shield that has a first end and a second end that is disposed opposite said first end. The external shield has an interior section, and the interior section has a diameter that accommodates the probe to be inserted into the interior section of the external shield. Additionally, at least one of the first end or the second end of the shield is open such that the housing of the probe is freely movable within and, optionally, outside of at least a portion of the external shield.

In yet another embodiment, the housing of the probe is movable for at least one of a lateral movement, a longitudinal movement, or a transverse movement within and, optionally, outside at least a portion of the external shield.

In another embodiment, the probe is in communication with a movement element for controlling said lateral, or longitudinal, or transverse movements of the probe within and, optionally, outside at least a portion of the external shield.

In yet another embodiment, the positron emission tomography sensor of the probe is positioned on a support board within the housing.

In another embodiment, the probe includes a biopsy gun attached to the external shell of the housing of the probe, and the biopsy gun is equipped with a biopsy needle.

In yet another embodiment, a mobile imaging system is provided comprising a bed for accommodating a patient, an open rotating gantry mounted around said bed and mobile with respect to said bed, a positron emission tomography imager having at least one mechanically separate positron emission tomography detector head secured to the rotating gantry above the bed and optionally at least one separate positron emission tomography detector head secured to the rotating gantry below the bed, wherein each of the detector heads are capable of angular rotation with respect to the bed to provide full angular projective sampling of a target organ of a patient lying on the bed, a probe comprising a housing having an external shell and an interior space, and a positron emission tomography sensor located within the interior space of the housing, wherein the probe is partially transmissive to a substantial fraction of 511 keV annihilation gamma rays, an electronic sensor positioning system located either on the exterior of the housing of the probe or within the interior of the housing and on or within each of the positron emission tomography detector heads such that the electronic sensor positioning system is in communication with said positron emission detector heads and the positron emission tomography sensor of the probe for spatially co-registering the probe to each detector head and for controlling an absolute and relative positioning of the probe, the positron emission tomography imager, and a target organ of a patient, and a high speed data acquisition computer system for collecting data from the positron emission sensor of the probe and the positron emission tomography imager.

In yet another embodiment, the mobile imaging system includes wherein the positron emission tomography sensor of the probe is disposed within the interior of the housing such that it is rotatable about an axis of rotation.

In another embodiment, the probe of the imaging system includes a positron emission photoarray and a positron emission detection electronics each in juxtaposition to and in communication with the positron emission sensor.

In yet another embodiment, the detector heads of the imaging system are capable of being operated in a static mode in which each of the detector heads are fixed in position with respect to a target organ of a patient lying on the bed, or in a dynamic mode in which each of the detector heads are rotated with respect to the target organ of the patient lying on the bed to provide full angular projective sampling of the target organ for enhanced tomographic 3D reconstruction, and wherein the detector heads can be rotated to a new viewing angle with respect to the target organ and then operated in the static mode to better view the target organ of the patient lying on the bed and to optimize positron emission tomographic 3D spatial resolution.

In another embodiment, the rotating gantry of the imaging system enables 360 degree angular sampling in a 3D imaging mode with the probe and the positron emission tomography imager.

In yet another embodiment, the positron emission tomography sensor of the probe of the imaging system is positioned on a support board within the housing of the probe.

In another embodiment, a method for evaluating a target organ of a patient is provided comprising injecting a patient with an imaging agent, providing a mobile imaging system comprising a bed for accommodating a patient, an open rotating gantry mounted around the bed and mobile with respect to the bed, a positron emission tomography imager having at least one mechanically separate positron emission tomography detector head secured to said rotating gantry above the bed and optionally at least one separate positron emission tomography detector head secured to the rotating gantry below the bed, wherein each of the detector heads are capable of angular rotation with respect to the bed to provide full angular projective sampling of a target organ of a patient lying on the bed, a probe comprising a housing having an external shell and an interior space, and a positron emission tomography sensor located within the housing, wherein the probe is partially transmissive to a substantial fraction of 511 keV annihilation gamma rays, and an electronic sensor positioning system located either on the exterior of the housing of the probe or within the interior of the housing and on or within each of said positron emission tomography detector heads such that the electronic sensor positioning system is in communication with the positron emission detector heads and the positron emission tomography sensor of the probe for spatially co-registering the probe to each detector head and for controlling an absolute and relative positioning of the probe, the positron emission tomography imager, and a target organ of a patient, and a high speed data acquisition computer system for collecting data simultaneously from the positron emission sensor of the probe and the positron emission tomography image, positioning the patient on the bed of the imaging system; and operating the imaging system such that the imaging system is positioned to scan a target organ of the patient.

In yet another embodiment, the method for evaluating a target organ of a patient includes positioning a biopsy gun on the external shell of the housing of the probe for conducting a biopsy of the target organ.

The additional features and advantage of the disclosed invention is set forth in the detailed description which follows, and will be apparent to those skilled in the art from the description or recognized by practicing the invention as described, together with the claims and appended drawings.

### BRIEF DESCRIPTION OF THE DRAWINGS

The foregoing aspects, uses, and advantages of the present invention will be more fully appreciated as the same becomes better understood from the following detailed description of the present invention when viewed in conjunction with the accompanying figures.

FIG. 1A shows an example of a stand-alone prostate PET imager with two panels and an endorectal PET probe of this invention

FIG. 1B shows the effect of time of flight (TOF) on the reconstructed tomographic volume.

FIG. 2 shows another example of the dedicated prostate PET imager using two pairs of panel modules and the endorectal PET probe of this invention, wherein the PET panels are as wide as the width of the patient's body.

FIG. 3 shows another example of implementation (top view looking down upon the patient), with the supine breast cancer patient and a panel insert PET module placed next to the patient's breast and inside the ring PET imager surrounding the patient.

FIG. 4 shows a preferred embodiment of the probe of the present invention inserted into the rectum of a patient.

FIG. 5 shows a patient on his side with the probe of this invention inserted into the rectum.

FIG. 6 shows an embodiment of this invention wherein a PET imaging detector is placed in front of the patient.

FIG. 7 shows the probe of this invention equipped with an optional biopsy gun for performing a biopsy if after identifying suspicious lesions based upon the PET results concerning elasticity characteristics.

FIG. 8 shows an embodiment of this invention wherein the probe having the PET sensor of the probe that is further enveloped in the external shield. The external shield having the probe of this invention is inserted into the rectum of a patient and two PET imaging panels placed stereotactically above the patient and operating in co-incidence with the PET sensor element of the probe of this invention.

FIG. 9 shows a preferred embodiment of the imaging system of this invention having PET imaging detectors positioned both above and below the patient and wherein the probe of this invention is disposed within an optional external shield is inserted into the rectum of a patient near the prostate gland.

### DETAILED DESCRIPTION OF THE INVENTION

The positron emission tomography probe and imaging system of this invention provide significant improvement over existing devices and methods to obtain evaluations of a target organ of a patient, a biopsy of the target organ, and to perform localized surgery of the target organ. The target organ may be, for example, but not limited to the prostate gland of a male patient, a gynecological anatomical structure of a female patient (vagina, cervix, uterus, etc.), or the colon of a patient, or other anatomical structure of a patient wherein an endoscopic probe is utilized.

The present invention provides an improvement to PET imaging by simultaneous imaging using a PET imager (of practically any design) and the high-resolution small compact probe(s) of the instant invention, as described herein, that are placed strategically inside the imaging volume of the PET scanner. Those persons skilled in the art appreciate that the prior proposed approach is to perform imaging with and without known PET probes separately and then combine the imaging results. (The known inserts [probes] could be also multimodal, for example combining PET/Ultrasound probes with temperature sensors, etc. on board.) While the PET scanner can provide broader view of the organ or region, the PET insert (probe) of the present invention operating with the PET scanner provides more accurate, typically higher resolution imaging only of the limited region of interest in the broader imaging scene provided by the PET scanner alone. In the present invention, both imaging modes can be performed simultaneously, with the ability to make almost automatic co-registration of the two images. Co-registration or fusion of the two imaging sets of data is crucial in providing useful information and guidance to the radiologist and surgeon.

Typically, as the standard known in the art practice, PET detector modules, inserts or probes are designed to absorb maximum fraction of annihilation 511 keV gamma rays impinging on them, as this is beneficial to the operation of the known PET systems with probes, by increasing the efficiency of operation. Therefore, in the images acquired with these known in the art PET scanners, such inserts or probes that are inserted inside the PET imaging volumes of any known PET scanners would be seen as mostly opaque objects hiding the PET image details of the tissue sections in front and behind them in the line of view. The present invention discloses PET inserts or probes that they can be sufficiently “seen through” to allow visibility of the tissue in line (in front or behind) with these probes or inserts. The partially transmitting probes of the present invention have by design a lower detection efficiency, however, the advantages from the simultaneous imaging provided by the method and imaging system of the present invention employing the probes of the present invention are advantages over the background art imaging systems.

In a preferred embodiment of this invention, the probe and imaging system and method of this invention is useful to guide prostate biopsy/surgery with high resolution PET (Positron Emission Tomography) probe imaging in an endorectal device. The probe of this invention is preferably made of a thinner partially transmissive material which improves spatial resolution of the PET probe plus probe insert system, as the depth of interaction effect, typically blurred spatial resolution when thicker absorbing layers of the scintillator material are used, becomes less important. This probe of the present invention is partially transmissive to a substantial fraction of the 511 keV annihilation gamma rays so that they can be detected by the PET scanner (detector) (with probe ignored), and simultaneous imaging by the PET scanner (detector) alone and PET scanner (detector) plus insert/probe. As used herein, the term “substantial” is defined as 50 percent (%) or greater. Only the stopped fraction of the annihilation gammas in the insert/probe contributes to the latter imaging.

The probe and imaging system of the invention is a novel dedicated high resolution probe system wherein the probe is partially transmissive to a substantial fraction of 511 keV annihilation gamma rays. Optionally, the probe and imaging system of this invention include a temperature probe, position and angle locator, as well as enhancements, described herein, to the basic operational parameters.

In a preferred embodiment of this invention, the method of the present invention utilizes a probe and the imaging system so that prostate biopsy can be performed accurately, which at the present time many of such biopsies of the prostate are poor at best.

The imaging system comprises the probe of this invention, operating with an external coincident PET module or set of modules in different configurations that will provide the metabolic information related to the biological state of the target organ, such as for example, the prostate gland, and specifically about the presence of any cancerous structures exhibiting increased metabolic activity. In addition to cancer diagnosis, the PET prostate probe and imaging system can be used in biopsy and in surgical guidance.

This invention provides a probe comprising a housing having an external shell and an interior space and a positron emission tomography sensor located within the interior space of the housing. The probe is partially transmissive to a substantial fraction of 511 keV annihilation gamma rays.

In a preferred embodiment, the positron emission tomography sensor of the probe is disposed within said interior of said housing such that it is rotatable about an axis of rotation.

In another embodiment, the probe includes a positron emission photoarray and a positron emission detection electronics each in juxtaposition to and in communication with the positron emission sensor.

In yet another embodiment, the probe includes an electronic sensor positioning system located either on the exterior of the housing of the probe or within the interior of the housing, and the electronic sensor positioning system is in communication with an outside positron emission tomography imager.

In another embodiment, the probe includes an external shield that has a first end and a second end that is disposed opposite said first end. The external shield has an interior section, and the interior section has a diameter that accommodates the probe to be inserted into the interior section of the external shield. Additionally, at least one of the first end or the second end of the shield is open such that the housing of the probe is freely movable within and, optionally, outside of at least a portion of the external shield.

In yet another embodiment, the housing of the probe is movable for at least one of a lateral movement, a longitudinal movement, or a transverse movement within and, optionally, outside at least a portion of the external shield.

In another embodiment, the probe is in communication with a movement element for controlling said lateral, or longitudinal, or transverse movements of the probe within and, optionally, outside at least a portion of the external shield.

In yet another embodiment, the positron emission tomography sensor of the probe is positioned on a support board within the housing.

In another embodiment, the probe includes a biopsy gun attached to the external shell of the housing of the probe, and the biopsy gun is equipped with a biopsy needle.

In yet another embodiment, a mobile imaging system is provided comprising a bed for accommodating a patient, an open rotating gantry mounted around said bed and mobile with respect to said bed, a positron emission tomography imager having at least one mechanically separate positron emission tomography detector head secured to the rotating gantry above the bed and optionally at least one separate positron emission tomography detector head secured to the rotating gantry below the bed, wherein each of the detector heads are capable of angular rotation with respect to the bed to provide full angular projective sampling of a target organ of a patient lying on the bed, a probe comprising a housing having an external shell and an interior space, and a positron emission tomography sensor located within the interior space of the housing, wherein the probe is partially transmissive to a substantial fraction of 511 keV annihilation gamma rays, an electronic sensor positioning system located either on the exterior of the housing of the probe or within the interior of the housing and on or within each of the positron emission tomography detector heads such that the electronic sensor positioning system is in communication with said positron emission detector heads and the positron emission tomography sensor of the probe for spatially co-registering the probe to each detector head and for controlling an absolute and relative positioning of the probe, the positron emission tomography imager, and a target organ of a patient, and a high speed data acquisition computer system for collecting data from the positron emission sensor of the probe and the positron emission tomography imager.

In yet another embodiment, the mobile imaging system includes wherein the positron emission tomography sensor of the probe is disposed within the interior of the housing such that it is rotatable about an axis of rotation.

In another embodiment, the probe of the imaging system includes a positron emission photoarray and a positron emission detection electronics each in juxtaposition to and in communication with the positron emission sensor.

In yet another embodiment, the detector heads of the imaging system are capable of being operated in a static mode in which each of the detector heads are fixed in position with respect to a target organ of a patient lying on the bed, or in a dynamic mode in which each of the detector heads are rotated with respect to the target organ of the patient lying on the bed to provide full angular projective sampling of the target organ for enhanced tomographic 3D reconstruction, and wherein the detector heads can be rotated to a new viewing angle with respect to the target organ and then operated in the static mode to better view the target organ of the patient lying on the bed and to optimize positron emission tomographic 3D spatial resolution.

In another embodiment, the rotating gantry of the imaging system enables 360 degree angular sampling in a 3D imaging mode with the probe and the positron emission tomography imager.

In yet another embodiment, the positron emission tomography sensor of the probe of the imaging system is positioned on a support board within the housing of the probe.

In another embodiment, a method for evaluating a target organ of a patient is provided comprising injecting a patient with an imaging agent, providing a mobile imaging system comprising a bed for accommodating a patient, an open rotating gantry mounted around the bed and mobile with respect to the bed, a positron emission tomography imager having at least one mechanically separate positron emission tomography detector head secured to said rotating gantry above the bed and optionally at least one separate positron emission tomography detector head secured to the rotating gantry below the bed, wherein each of the detector heads are capable of angular rotation with respect to the bed to provide full angular projective sampling of a target organ of a patient lying on the bed, a probe comprising a housing having an external shell and an interior space, and a positron emission tomography sensor located within the housing, wherein the probe is partially transmissive to a substantial fraction of 511 keV annihilation gamma rays, and an electronic sensor positioning system located either on the exterior of the housing of the probe or within the interior of the housing and on or within each of said positron emission tomography detector heads such that the electronic sensor positioning system is in communication with the positron emission detector heads and the positron emission tomography sensor of the probe for spatially co-registering the probe to each detector head and for controlling an absolute and relative positioning of the probe, the positron emission tomography imager, and a target organ of a patient, and a high speed data acquisition computer system for collecting data simultaneously from the positron emission sensor of the probe and the positron emission tomography image, positioning the patient on the bed of the imaging system; and operating the imaging system such that the imaging system is positioned to scan a target organ of the patient.

In yet another embodiment, the method for evaluating a target organ of a patient includes positioning a biopsy gun on the external shell of the housing of the probe for conducting a biopsy of the target organ.

A preferred embodiment of the method of the present invention is set forth below. Before a prostate biopsy is performed, the method of evaluating the prostate gland and any region of interest (ROI) thereof is performed. The patient will be injected systemically into a vein with a PET imaging agent targeting the prostate or a ROI of the prostate know to have cancer. During this procedure, the PET probe operating with the external PET imager modules (see attached Figures) will be used to scan the region of the prostate for any signs of unusually high uptake of the PET imaging agent, reflecting the presence of a potentially cancerous structure/lesion. This in turn can provide guidance for biopsy and for surgery if the surgery needed. If the patient undergoes surgery for a positive biopsy result, the prostate will be examined closely for correlation with the PET finding. As noted above, for example, the PET imaging system can have also applications in gynecological exams and potentially also in colon exams.

FIG. 1A shows an embodiment of the probe and imaging system of the present invention of a close to optimal stand-alone prostate PET imager with two PET detector panels and an endorectal PET probe. All imaging modules (panels and probe) have on-board positioning system installed so that their relative positions will be, at all times, known and recorded. The two PET panels operate as a high resolution PET imager. To obtain angular sampling, viewing from all angles, the panels are coregistered and rotated together on a computer controlled gantry to obtain the full set of projective data, to allow for tomographic 3D reconstruction. Those persons skilled in the art understand that, typically, the currently used methodology of organ evaluation during the full PET imaging phase, dictates that the currently known endorectal PET probe is removed to avoid interference of the probe during imaging. Next, the probe is inserted and the second imaging session is performed using the probe and the top PET panel detector module. During that second part, the bottom imaging panel is not used. In contrast to the current methodology, the present invention provides a method wherein the PET probe of this invention is inserted all the time, and fraction of the lines of response (LORs) is traversing the probe. Due to relatively high stopping power of the probe compared to the human body, a substantial fraction of the 511 keV annihilation gamma rays going along these lines will be absorbed in the probe and will not reach the bottom PET panel. A substantial fraction is defined as 50 percent or greater. However, with proper adjustment of the parameters the fraction of the total number of the LORs reaching the bottom panel will be still sufficient to produce good quality full PET image, even if showing shadow from the probe absorption of gammas. The main advantage of this partially absorbing/transmitting probe of this invention is that no movement of the probe, and no associated movement of the prostate will take place, and therefore the image coregistration of the full PET image using only the PET panels, and the top panel plus the probe high resolution imaging, is relatively simple. Additionally, with imaging done at the same time, the overall procedure of this invention takes less time and is less expensive. FIG. 1B shows the effect of time of flight (TOF) on the reconstructed tomographic volume. Those persons skilled in the art will appreciate that the present invention's advantage is that the size of the PET panels can be smaller to cover the shrunk (through enacted TOF condition) required imaging volume. With no TOF function in place, the good practice assuring good quality tomographic reconstruction is to use imaging modules as wide as the width of the patient's body, as shown in FIG. 2.

FIG. 2 shows another example of the dedicated prostate PET imager using two pairs of panel modules and the endorectal PET probe of this invention. Examples of possible panel detector sizes and the geometry are shown in FIG. 2. Angular coverage and efficiency of this embodiment of the present invention's imaging system are higher than the system of FIG. 1, however complexity and cost are also increased.

FIG. 3 shows another example of implementation of this invention, with the supine breast cancer patient and a panel insert PET module placed next to the patient's breast and inside the ring PET imager surrounding the patient. Imaging with PET imager can be performed at the same time when imaging with the panel plus section of the PET imager ring is performed. Some LORs represent the PET ring imaging, some are LORs in the PET ring imager that traverse the panel insert module, and some LORS represent imaging performed between the panel insert and the corresponding section of the PET ring, opposite to the panel and with the emitting hot spot in the breast in between the panel and the ring.

FIG. 4 shows a preferred embodiment of the probe of the present invention inserted into the rectum of a patient. FIG. 4 shows the probe inserted into the rectum during the PET imaging phase. FIG. 5 shows a patient on his side with the probe inserted into the rectum. By optimizing the angle of the probe, the PET sensor can be better aligned with the prostate gland. One or more PET imaging detector or detectors (not shown in FIG. 5, but shown in FIG. 6) is placed in front of the patient. The PET imaging detector operates in co-incidence with the probe.

FIG. 7 shows the probe of this invention equipped with an optional biopsy gun for performing a biopsy if after identifying suspicious lesions based upon the PET results concerning elasticity characteristics.

A preferred embodiment of the probe of the present invention includes (i) a PET sensor (scintillator+SiPM [silicon photomultiplier] photodetector), (ii) a 6 degrees of freedom (3 coordinates and 3 angles) positioning sensor with readout, (iii) Fusion algorithms and software, fusing the PET modalities in 3D and in 2D projections for viewing and guidance, and optionally (iv) one or more temperature sensors with bias voltage feedback for the PET probe. Further, the probe of the present invention may optionally include a fast signal electronics implementation of the PET sensor for the Time of Flight (TOF) capability.

The preferred technology of the PET probe is a combination of compact Silicon Photomultipliers (SiPMs) and pixellated scintillators. The scintillators detect the 511 keV annihilation gammas from positron emissions in the prostate and surroundings and convert the detected energy into scintillation signals which are in turn detected in the SiPM photodetectors.

The external PET panel detectors can be built using different combinations of photodetectors and scintillators. In a preferred embodiment of the PET detectors, the panel detectors are position sensitive photomultipliers and pixellated scintillators. The outer PET detectors can take different forms, from full rings to simple panels.

FIG. 8 shows an embodiment of this invention wherein the probe having the PET sensor (not shown) that is further partially enveloped in the external shield (32) is inserted into the rectum of a patient and two PET imaging panels (40, 41) placed stereotactically above the patient and operating in co-incidence with the PET sensor element of the probe of this invention. To immobilize the prostate during the imaging system scan of this invention, the wall of the external shield (32) (having a larger diameter than the diameter of the probe of this invention) is used to immobilize the prostate during the whole imaging system procedure or method of the present invention. During the imaging system scan, the probe of this invention is moved inside the external shield longitudinally and transversely to cover the necessary volume of the prostate for the positron emission tomography scan.

FIG. 9 shows a preferred embodiment of the imaging system and probe of the present invention. FIG. 9 does not show the target organ and probe to scale and the drawing should be treated as showing the invention elements. In FIG. 9, the imaging system of this invention shows that PET detectors are made up of sixteen (16), in this example, external PET detector modules divided into two sectors: top (50) and bottom (52), placed on an approximately cylindrical surface, and the probe of this invention having the PET sensor. The probe (10) is placed inside the optional external shield (32), as described herein, and the shield (32) is then placed endorectally into the patient under the prostate (46). The probe's size permits that at any position of the probe (10), only a fraction of the lines of response (44) of back-to-back coincident 511 keV annihilation gamma ray pairs between the front external PET detector modules and the probe of this invention, is recorded. The probe of this invention has a 6-parameter (3 coordinates and 3 angles) position probe that monitors and records positioning of the PET probe of this invention relative to the outside PET detector modules of the imaging system of this invention. Optionally, a temperature probe is included with the PET probe of this invention and the positioning probe to compensate the temperature-sensitive SiPMs.

The optional external shield (32) is placed in the patient (rectum or other cavity) in a constant position at all times during the imaging system scan of this invention. The probe of this invention is inserted into the external shield and then the probe is moved inside the external shield during the scan and method of this invention. The presence of the external shield (32) is exerting constant and stable pressure on the prostate and surrounding tissues and stabilizing the target organ and surrounding tissues during the method/scan of the present invention.

### Construction of the PET Sensor

A working PET sensor based upon an array of 4×10 MPCC (multipixel photon counter) SiPMs was obtained from and is commercially available from Hamamatsu, with approximately 15 mm×45 mm active FOV. This particular sensor was equipped with 16×18 mm scintillator array defining this as the active size of the PET probe. Amplifier and connector banks are in the handle region of the probe.

The size of the probe of this invention is, for example but not limited to, 3 cm wide by 2 cm tall. The photodetector sensor is built out of 72 MPPCs units commercially available from Hamamatsu arranged in a 6×12 array. The 3×3×mmm MPPC units were spaced at 5 mm center to center distance. In the center of the probe is the input stage electronics (amplifiers) and at the right is the bank of cable connectors matching with three small profile flat cables.

### PET Imaging Agents

While this invention is not focusing on the imaging agents used to visualize the prostate cancer in PET modality, the issue of proper selection of PET imaging agent is important, as prostate cancer, unlike most of other cancers, is not avidly absorbing glucose analog—¹⁸F-Flurodeoxyglucose (FDG}—used in the standard PET scans. In a recent paper, Mullani et al., First-Pass 18F-FDG PET of Blood Flow, The Journal of Nuclear Medicine, Volume 49, No. 4, April, 2008, the authors propose that even in the case of prostate cancer FDG will provide valid indication of prostate cancer, if used in the first-pass mode. Imaging is then performed in the first 2 minutes post-injection (first-pass blood flow procedure) and then repeated as a standard glucose uptake scan about 45-60 minutes later, so that two comparative images are obtained allowing for better identification of prostate cancer than with the standard glucose uptake image only. Therefore, we are specifically mentioning this unique method, as FDG is readily available in medical facilities and this fact will be a major enabler of the present invention, while also including other imaging agents such as Choline, and other prostate cancer specific imaging agents under development. Some examples are listed below (but are not limited to this list):

### [^{64/62/60}Cu]ATSM/PTSM

¹⁸F, ¹¹C-labeled choline analogs
[¹¹ C] acetate

### [¹⁸F]FMAU

16b-[¹⁸F]fluoro-5a-dihydrotestosterone, etc.

The present invention relates generally to a device that functions to provide accurate localization of a target tumor or organ. The probe includes a positron emission tomography (PET) component to provide metabolic information related to the biological state of the target tumor or organ. In particular, when used to image the prostate gland of a male patient, the system of this invention may be used to specifically detect the presence of cancerous structures in the prostate, and may be adapted to identify cancerous structures showing increased metabolic activity. The imaging information obtained by using the present imaging system may be used to provide guidance for a biopsy procedure, for example, or for other medical procedures requiring surgical intervention.

It is understood by one skilled in the art that a standard positron emission tomography scanner typically offers a spatial resolution of only about 5 mm, at best. This precision is not adequate to show details of uptake in small organs such as the prostate gland. Accordingly, the present invention provides a combination of high-resolution positron emission tomography with new imaging markers. This novel combination provides for a highly improved molecular imaging system and method for the detection of prostate cancer.

Recent developments in the field of compact Silicon Photomultipliers (SiPMs), have enabled the fabrication of a positron emission tomography module that operates with high resolution PET detectors to provide a resolution on the order of one millimeter. Such resolution in the present mobile imaging system enables the imaging of the prostate gland.

In an exemplary embodiment of the present invention, a positron emission tomography sensor may comprise compact silicon photomultipliers and pixellated scintillators. Scintillator materials may comprise CsI(TI), CsI(Na), GSO, NaI(TI), and LaBr3. A tungsten composite may be used for a collimator material. The scintillators function to: (i) detect incident radiation comprising 511 KeV annihilation gammas from positron emissions in the prostate gland, and (ii) convert the incident radiation conversion into scintillation signals. The scintillation signals are, in turn, detected by the silicon photomultipliers and provided to detection/control electronics.

### Reduction to Practice

Initially, both the mini PET probe and the limited tomography PET system were used with the PET probe inserted in the field of view of the PET scanner. Two 20×15 cm panel PET modules were mounted on a computer controlled research rotating gantry. A resolution phantom was used during tests with the miniature PET probe. Also, a torso phantom was used with the second PET probe, the PET panel detector and the prostate phantom. The PET imager comprised two panel detectors on the rotating gantry and the PET probe was in place at the same time and at the same imaging geometry, and used for sequential imaging. The simultaneous imaging was found to be feasible.

### Results

The miniature probe's shadow could be seen in the PET imaging between the two PET panels when placed in front of a flood box phantom. Only a flood box was present, the resolution phantom was removed for a measurement. The local drop in counting rate was only about 25%, which permits for proper operation of the PET scanner.

Planar PET images from the two-panel PET scanner and from the probe operating with the top panel were observed. The details of the resolution phantom not separated in the PET image (2.5 mm spatial resolution) are separated (1.1 mm resolution) due to excellent spatial resolution of the probe (0.7 mm).

Those persons skilled in the art will understand that changes could be made to the embodiments described above without departing from the inventive concept of the probe, the imaging system, and methods of the present invention. The accompanying drawings are included to provide a further understanding of various features and embodiments of the probe, imaging system, and methods of the invention which, together with their description serve to explain the principles and operation of the invention set forth herein. It is understood, therefore, that this invention is not limited to the particular embodiments disclosed, but is intended to cover modifications that are within the spirit and scope of the invention, as defined by the appended claims.

We claim:

1. A probe comprising:
a housing having an external shell and an interior space; and
a positron emission tomography sensor located within said interior space of said housing, wherein said probe is partially transmissive to a substantial fraction of 511 keV annihilation gamma rays.

2. The probe of claim 1 wherein said positron emission tomography sensor is disposed within said interior of said housing such that it is rotatable about an axis of rotation.

3. The probe of claim 1 including a positron emission photoarray and a positron emission detection electronics each in juxtaposition to and in communication with said positron emission sensor.

4. The probe of claim 1 including an electronic sensor positioning system located either on said exterior of said housing of said probe or within said interior of said housing wherein said electronic sensor positioning system is in communication with an outside positron emission tomography imager.

5. The probe of claim 1 including an external shield that has a first end and a second end that is disposed opposite said first end, said external shield having an interior section, said interior section having a diameter that accommodates said probe to be inserted into the interior section of said external shield, and wherein at least one of said first end or second end of said shield is open such that said housing of said probe is freely movable within and outside of at least a portion of said external shield.

6. The probe of claim 5 including wherein said housing of said probe is movable for at least one of a lateral movement, a longitudinal movement, or a transverse movement within and outside at least a portion of said external shield.

7. The probe of claim 6 including wherein said probe is in communication with a movement element for controlling said lateral, or longitudinal, or transverse movements of said probe within and outside at least a portion of said external shield.

8. The probe of claim 1 wherein said positron emission tomography sensor is positioned on a support board within said housing.

9. The probe of claim 1 including a biopsy gun attached to the external shell of said housing of said probe, said biopsy gun equipped with a biopsy needle.

10. A mobile imaging system comprising:
a bed for accommodating a patient;
an open rotating gantry mounted around said bed and mobile with respect to said bed;
a positron emission tomography imager having at least one mechanically separate positron emission tomography detector head secured to said rotating gantry above said bed and optionally at least one separate positron emission tomography detector head secured to said rotating gantry below said bed, wherein each of said detector heads are capable of angular rotation with respect to said bed to provide full angular projective sampling of a target organ of a patient lying on said bed;
a probe comprising a housing having an external shell and an interior space, and a positron emission tomography sensor located within said interior space of said housing, wherein said probe is partially transmissive to a substantial fraction of 511 keV annihilation gamma rays;
an electronic sensor positioning system located either on said exterior of said housing of said probe or within said interior of said housing and on or within each of said positron emission tomography detector heads such that said electronic sensor positioning system is in communication with said positron emission detector heads and said positron emission tomography sensor of said probe for spatially co-registering said probe to each detector head and for controlling an absolute and relative positioning of said probe, said positron emission tomography imager, and a target organ of a patient; and
a high speed data acquisition computer system for collecting data from said positron emission sensor of said probe and said positron emission tomography imager.

11. The mobile imaging system of claim 10 wherein said positron emission tomography sensor of said probe is disposed within said interior of said housing such that it is rotatable about an axis of rotation.

12. The imaging system of claim 10 wherein said probe includes a positron emission photoarray and a positron emission detection electronics each in juxtaposition to and in communication with said positron emission sensor.

13. The imaging system of claim 10 wherein said detector heads are capable of being operated in a static mode in which each of said detector heads are fixed in position with respect to a target organ of a patient lying on said bed, or in a dynamic mode in which each of said detector heads are rotated with respect to the target organ of the patient lying on said bed to provide full angular projective sampling of the target organ for enhanced tomographic 3D reconstruction, and wherein said detector heads can be rotated to a new viewing angle with respect to said target organ and then operated in said static mode to better view the target organ of the patient lying on said bed and to optimize positron emission tomographic 3D spatial resolution.

14. The imaging system of claim 10 wherein said rotating gantry enables 360 degree angular sampling in a 3D imaging mode with said probe and said positron emission tomography imager.

15. The imaging system of claim 10 wherein said positron emission tomography sensor of said probe is positioned on a support board within said housing of said probe.

16. A method for evaluating a target organ of a patient comprising:
injecting a patient with an imaging agent;
providing a mobile imaging system comprising:
a bed for accommodating a patient, an open rotating gantry mounted around said bed and mobile with respect to said bed, a positron emission tomography imager having at least one mechanically separate positron emission tomography detector head secured to said rotating gantry above said bed and optionally at least one separate positron emission tomography detector head secured to said rotating gantry below said bed, wherein each of said detector heads are capable of angular rotation with respect to said bed to provide full angular projective sampling of a target organ of a patient lying on said bed, a probe comprising a housing having an external shell and an interior space, and a positron emission tomography sensor located within said housing, wherein said probe is partially transmissive to a substantial fraction of 511 keV annihilation gamma rays, and an electronic sensor positioning system located either on said exterior of said housing of said probe or within said interior of said housing and on or within each of said positron emission tomography detector heads such that said electronic sensor positioning system is in communication with said positron emission detector heads and said positron emission tomography sensor of said probe for spatially co-registering said probe to each detector head and for controlling an absolute and relative positioning of said probe, said positron emission tomography imager, and a target organ of a patient, and a high speed data acquisition computer system for collecting data simultaneously from said positron emission sensor of said probe and said positron emission tomography image;
positioning said patient on said bed of said imaging system; and
operating said imaging system such that said imaging system is positioned to scan a target organ of said patient.

17. The method of claim 16 including positioning a biopsy gun on said external shell of said housing of said probe for conducting a biopsy of said target organ.
